(19) United States
(12) Patent Application Publication
(10) Pub. No.: US 2009/0275130 A1
(43) Pub. Date: Nov. 5, 2009

(54) BIOMIMETIC NUCLEIC ACIDS

(75) Inventors: Stephen Navran, Houston, TX (US); Ulrich Strych, Houston, TX (US); George Jackson, Pearland, TX (US)

Correspondence Address: BIO TEX, INC., 8058 EL RIO STREET, HOUSTON, TX, 77054, US

(73) Assignees: BIOTEX, INC., Houston, TX (US); SYNTHECON, INC., Houston, TX (US)

(21) Appl. No.: 12/434,644

(22) Filed: May 2, 2009

Related U.S. Application Data
(60) Provisional application No. 61/050,016, filed on May 2, 2008.

(51) Int. Cl. C40B 30/04; C07H 21/04; C12N 5/08

(52) U.S. Cl. 435/366; 506/9; 536/23.1

(57) ABSTRACT

The present invention is directed to nucleic acids with biomimetic properties and methods for producing said nucleic acids. In particular, this invention relates to nucleic acids exhibiting biomimetic properties in relation to proteins such as growth factors, hormones and/or other cell signaling proteins. Biomimetic properties may generally be defined as interactive ability in the same and/or similar manner as another biological molecule. This may, for example, include interacting with a ligand-binding biomolecule, such as a cell signaling receptor, in a manner similar to a native ligand. In the case of a signaling receptor, such biomimetic nucleic acids may in general act as an agonist or an antagonist to the given receptor. They may further act in competition to a native ligand.

### CROSS-REFERENCE TO RELATED APPLICATIONS

This application claims the benefit of U.S. provisional patent application Ser. No. 61/050,016, filed May 2, 2008, entitled “BIOMIMETIC NUCLEIC ACIDS”, the entire contents of which are hereby incorporated by reference.

### SEQUENCE LISTING

The nucleotide sequences 5′-ataccagcttattcaattGGCAAGGGGTAGACACGCGGCGCGGGACCGGGAGCCGACAa gatagtaagtgcaatct-3′,5′-agatagtaagtgcaatctGTTAAGTTTGACTATAACAACCCGGACCTGTTATTCGGGGA ATTGAATAAGCTGGTAT-3′, 5′-ataccagcttattcaattGGCAAGGGgtagacACGCGGCGCGGGACCGGGAGCCGACAa gatagtaagtgcaatctGTTAAGTTTGACTATAACAACCCGGACCTGTTATTCGGGGAA TTGAATAAGCTGGTAT-3′, 5′-ataccagcttattcaattGGCCAGGCACTAACTAGTTGGCCGCATTAAAGACCTAATGa gatagtaagtgcaatct-3′,5′-agatagtaagtgcaatctATACGAGCGTGATTATCAATCCTCGTACACCGGGTACTGGA ATTGAATAAGCTGGTAT-3′, and 5′-ataccagcttattcaattGGCCAGGCACTAACTAGTTGGCCGCATTAAAGACCTAATGa gatagtaagtgcaatctATACGAGCGTGATTATCAATCCTCGTACACCGGGTACTGGAA TTGAATAAGCTGGTAT-3′, titled SEQ ID NO 1, SEQ ID NO 2, SEQ ID NO 3, SEQ ID NO 4, SEQ ID NO 5, and SEQ ID NO 6, respectively, are hereby incorporated by reference to the ASCII text file entitled “P1017US01_SEQIDS.txt”, created May 1, 2009, of 2,499 bytes in size.

### FIELD OF THE INVENTION

This invention relates to nucleic acids with biomimetic properties and methods for producing said nucleic acids.

### BACKGROUND OF THE INVENTION

The potential for the use of stem cells in regenerative medicine has produced considerable excitement. Much of the recent research in stem cells has focused on the factors that control self-renewal or differentiation, in particular, soluble molecules. However, many studies have demonstrated that stem cell self-renewal cannot be maintained by soluble mediators alone, but rather depends on the microenvironment or niche consisting of stromal cells and extracellular matrix. While most of the molecules that regulate stem cell fate are unknown, it is clear that whether they are soluble, part of the extracellular matrix or on the surface of other cells in the niche, they all bind to receptors on the surface of the stem cell to activate signaling pathways that control the ultimate response, the stem cell fate. The identity of these molecules is now under intensive study.

Even assuming that all of the ligands that control self-renewal or differentiation of stem cells are discovered, utilizing them for large-scale production of stem cells for regenerative therapy remains challenging. Virtually all of these ligands are proteins which are difficult to produce even at research scale. Producing these reagents in quantities necessary for clinical applications will be prohibitively expensive.

### SUMMARY OF THE INVENTION

The present invention is directed to nucleic acids with biomimetic properties and methods for producing said nucleic acids. In one exemplary embodiment, for example, this invention relates to nucleic acids exhibiting biomimetic properties in relation to proteins such as growth factors, hormones and/or other cell signaling proteins. Biomimetic properties may generally be defined as interactive ability in the same and/or similar manner as another biological molecule(s). This may, for example, include interacting with a ligand-binding biomolecule, such as a cell signaling receptor, in a manner similar to a native ligand. In the case of a signaling receptor, such biomimetic nucleic acids may in general act as an agonist or an antagonist to the given receptor, for example. They may further act in competition to a native ligand, for example.

In one aspect of the present invention, biomimetic nucleic acids may be aptamers that are, or including but not limited to, single-stranded nucleic acid, such as, for example, single-stranded DNA (ssDNA), single-stranded RNA (ssRNA), and/or a combination thereof; at least a portion of double-stranded nucleic acid, such as, for example, double-stranded DNA (dsDNA), double-stranded RNA (dsRNA), and/or combinations thereof; modified nucleotides and/or other useful molecules, moieties, and/or other functional chemical components, or combinations thereof; or combinations thereof or similar.

In general, the biomimetic nucleic acids may bind with relatively high specificity to a given target and may further act in a functional manner, such as with agonist or antagonist activity. Further, the biomimetic nucleic acids may at least partially mimic the functional activity of a native biomolecule

In an exemplary embodiment, biomimetic nucleic acids may mimic the activity of cell growth, proliferation and/or differentiation signaling molecules. Such molecules may include, for example, growth factors, hormones, and/or any other appropriate signaling molecule. The biomimetic nucleic acids may then be utilized in place of said signaling molecule in, for example, cell culture. This may be useful in, for example, stem cell culture, where signaling molecules may be employed to maintain an undifferentiated state and/or promote undifferentiated proliferation of stem cells.

Biomimetic nucleic acids may be generated as aptamers utilizing selective propagation methods. In some exemplary embodiments, biomimetic nucleic acids may be generated as aptamers from large random libraries, for example, of nucleic acids, utilizing an iterative process called Systematic Evolution of Ligands by Exponential Enrichment (SELEX). Resultant aptamers may be further screened for a particular functional activity, such as, for example, agonist or antagonist activity against a cell signaling receptor. Such screening may be performed utilizing a native ligand for comparison of activity. Appropriate aptamers may then be produced on a large scale at a relatively low cost utilizing nucleic acid synthesis and/or other nucleic acid production methods, which may include cloning and/or fermentation methods.

In an exemplary embodiment, biomimetic nucleic acids may be selected utilizing a SELEX protocol which may include at least one selective displacement step. For example, candidate aptamers which may be bound to a target may be selectively displaced utilizing a competitive molecule. In one embodiment, aptamers may be selected for binding activity to a receptor molecule utilizing a native ligand for the receptor to selectively displace aptamers bound to the receptor molecule, for example, in the active site of the receptor molecule. This may be useful, for example, to aid in selecting aptamers for agonist or antagonist activity.

The present invention together with the above and other advantages may best be understood from the following detailed description of the exemplary embodiments and of the invention illustrated in the drawings.

### BRIEF DESCRIPTION OF THE FIGURES

FIG. 1 illustrates an example of whole-cell SELEX;

FIG. 1a illustrates whole-cell SELEX against stem cell factor receptors;

FIG. 2 illustrates an example of a log dose response curve;

FIG. 3 illustrates fold increase in cell proliferation from multiple additions; and

FIG. 4 shows a table of receptor-ligand pairs.

### DETAILED DESCRIPTION OF THE INVENTION

The detailed description set forth below is intended as a description of the presently exemplified device provided in accordance with aspects of the present invention and is not intended to represent the only forms in which the present invention may be practiced or utilized. It is to be understood, however, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices, compositions and materials similar or equivalent to those described herein can be used in the practice or testing of the invention, the exemplified methods, devices, compositions and materials are now described.

All publications mentioned herein are incorporated herein by reference for the purpose of describing and disclosing, for example, the compositions and methodologies that are described in the publications which might be used in connection with the presently described invention. The publications listed or discussed above, below and throughout the text are provided solely for their disclosure prior to the filing date of the present application. Nothing herein is to be construed as an admission that the inventors are not entitled to antedate such disclosure by virtue of prior invention.

The present invention is directed to nucleic acids with biomimetic properties and methods for producing said nucleic acids. In one exemplary embodiment, for example, this invention relates to nucleic acids exhibiting biomimetic properties in relation to proteins such as growth factors, hormones and/or other cell signaling proteins. Biomimetic properties may generally be defined as interactive ability in the same and/or similar manner as another biological molecule(s). This may, for example, include interacting with a ligand-binding biomolecule, such as a cell signaling receptor, in a manner similar to a native ligand. In the case of a signaling receptor, for example, such biomimetic nucleic acids may in general act as an agonist or an antagonist to the given receptor. They may, for example, further act in competition to a native ligand.

In one aspect of the present invention, biomimetic nucleic acids may be aptamers. An “aptamer” refers to a biomolecule that is capable of binding to a particular molecule of interest with high affinity and specificity. The binding of a target to an aptamer, which may be a nucleic acid such as RNA or DNA, or a combination thereof, or a peptide sequence, may generally derive from secondary and/or three-dimensional (3D) structures of the aptamer and the binding may also change the conformation and/or structure of the aptamer. This type of interaction, with a small molecule metabolite, for example, coupled with subsequent changes in aptamer function where the aptamer may be an RNA, may be referred to as a ‘riboswitch’. Aptamers may also include non-natural nucleotides, nucleotide analogs, non-natural amino acids and/or amino acid analogs. The method of selection may be by, but is not limited to, affinity chromatography and the method of amplification by reverse transcription (RT), polymerase chain reaction (PCR) and/or any other appropriate amplification method. Aptamers may include specific binding regions which may be capable of binding, attaching, and/or forming complexes with an intended target in an environment wherein other substances in the same environment may not bound, attached, and/or complexed to the aptamer. The specificity of the binding may be defined in terms of the comparative dissociation constants (Kd) of the aptamer for its target as compared to the dissociation constant of the aptamer for other materials in the environment or unrelated molecules in general. Typically, the Kd for the aptamer with respect to its target may be at least about 10-fold less than the Kd for the aptamer with unrelated material and/or accompanying material in the environment. In another example, the Kd may be at least about 50-fold less, in a further example, at least about 100-fold less, and in some exemplary examples at least about 200-fold less. A nucleic acid aptamer may typically be between about 10 and about 300 nucleotides in length, for example. In general, an aptamer may also be between about 30 and about 100 nucleotides in length. The terms “nucleic acid molecule” and “polynucleotide” may refer to deoxyribonucleotides or ribonucleotides and polymers thereof in either single- or double-stranded form. In general, the term may refer to nucleic acids containing known analogues of natural nucleotides which may have similar binding properties as the reference nucleic acid and may be metabolized in a manner similar to naturally occurring nucleotides. A particular nucleic acid sequence may also implicitly encompass conservatively modified variants thereof (e.g., degenerate codon substitutions) and/or complementary sequences, as well as the sequence. Degenerate codon substitutions may be achieved by generating sequences in which the third position of one or more selected (or all) codons may be substituted with mixed-base and/or deoxyinosine residues. Also included may be molecules that may have naturally occurring phosphodiester linkages as well as those that may have non-naturally occurring linkages, e.g., for stabilization purposes. The nucleic acid may be in any physical form, such as e.g., linear, circular, or supercoiled. The term nucleic acid may also be used interchangeably with oligonucleotide, gene, cDNA, and mRNA encoded by a gene.

Aptamers may be or include, but are not limited to, single-stranded nucleic acid, such as, for example, single-stranded DNA (ssDNA), single-stranded RNA (ssRNA), and/or a combination thereof; at least a portion of double-stranded nucleic acid, such as, for example, double-stranded DNA (dsDNA), double-stranded RNA (dsRNA), and/or combinations thereof; modified nucleotides and/or other useful molecules, moieties, and/or other functional chemical components, or combinations thereof; or combinations thereof or similar, as noted before.

In general, the biomimetic nucleic acids may bind with relatively high specificity to a given target and may further act in a functional manner, such as with agonist or antagonist activity. Further, the biomimetic nucleic acids may at least partially mimic the functional activity of a native biomolecule. In general, agonists may generally substantially enhance, activate and/or otherwise promote some function of a target molecule and an antagonist may in general substantially deactivate, and/or decrease a given function of a target molecule. For example, cell receptor molecule agonists may in general activate some signal transduction mechanism which may be coupled and/or related to the receptor. Also for example, an antagonist of a cell receptor may in general block some downstream function of the receptor, such as, for example, binding and/or complexing with the receptor in a manner that may not substantially activate a downstream mechanism and/or prevent the binding and/or complexing of an agonist, such as by competitive or suicide inhibition.

In general, modified nucleic acid bases may be utilized and may include, but are not limited to, 2′-Deoxy-P-nucleoside-5′-Triphosphate, 2′-Deoxyinosine-5′-Triphosphate, 2′-Deoxypseudouridine-5′-Triphosphate, 2′-Deoxyuridine-5′-Triphosphate, 2′-Deoxyzebularine-5′-Triphosphate, 2-Amino-2′-deoxyadenosine-5′-Triphosphate, 2-Amino-6-chloropurine-2′-deoxyriboside-5′-Triphosphate, 2-Aminopurine-2′-deoxyribose-5′-Triphosphate, 2-Thio-2′-deoxycytidine-5′-Triphosphate, 2-Thiothymidine-5′-Triphosphate, 2′-Deoxy-L-adenosine-5′-Triphosphate, 2′-Deoxy-L-cytidine-5′-Triphosphate, 2′-Deoxy-L-guanosine-5′-Triphosphate, 2′-Deoxy-L-thymidine-5′-Triphosphate, 4-Thiothymidine-5′-Triphosphate, 5-Aminoallyl-2′-deoxycytidine-5′-Triphosphate, 5-Aminoallyl-2′-deoxyuridine-5′-Triphosphate, 5-Bromo-2′-deoxycytidine-5′-Triphosphate, 5-Bromo-2′-deoxyuridine-5′-Triphosphate, 5-Fluoro-2′-deoxyuridine-5′-Triphosphate, and/or any other appropriate modified nucleic acid base. It may generally be understood that the nucleoside triphosphates (NTPs) listed above may generally refer to any appropriate phosphate of the modified base, such as additionally, for example, monophosphates (NMPs) or diphosphates (NDPs) of the base.

In an exemplary embodiment, biomimetic nucleic acids may mimic the activity of cell growth, proliferation and/or differentiation signaling molecules. Such molecules may include, for example, growth factors, hormones, and/or any other appropriate signaling molecule. Biomimetic nucleic acids may be generated as aptamers utilizing selective propagation methods. In exemplary embodiments, biomimetic nucleic acids may be generated as aptamers from large random libraries of, for example, nucleic acids, utilizing an iterative process generally referred to as Systematic Evolution of Ligands by Exponential Enrichment (SELEX) and any appropriate variations and/or modifications thereof. Resultant aptamers may be further screened for a particular functional activity, such as, for example, agonist or antagonist activity against a cell signaling receptor. Such screening may be performed utilizing a native ligand, such as a native agonist or antagonist, for comparison of activity. Comparison screening may include, for example, cell growth assays, proliferation assays, signal transduction assays, morphological assays, differentiation assays, expression assays and/or any other appropriate assays or combinations thereof. In general, any appropriate molecular biology and/or biochemical analysis and/or assay may be utilized in screening. Appropriate aptamers may then be produced on a large scale at a relatively low cost utilizing nucleic acid synthesis and/or other nucleic acid production methods, which may include cloning and/or fermentation methods. The biomimetic nucleic acids may be utilized in place of said signaling molecule in, for example, cell culture. This may be particularly useful in, for example, stem cell culture, where signaling molecules may be employed to maintain an undifferentiated state and/or promote undifferentiated proliferation of stem cells. This may also be useful in generally any situation where a signaling molecule may be utilized.

In general, generated aptamers may also be analyzed, such as by sequencing, sequence clustering, folding, conformation and/or shape determination, motif-identification, and/or by any other appropriate method of analysis or combination thereof. For example, after multiple rounds of selection in SELEX, particular sequence motifs and/or clusters may emerge as dominant. This may be useful, for example, in determining particular aptamer features that may play a substantial role in the binding activity of the aptamers.

In general, the SELEX method may include contacting a library of, for example, nucleic acids with at least one target, such as, for example, whole cell(s); target molecules, such as isolated and/or partially isolated receptor molecules; and/or any other appropriate target. In general, the members of the library that do not bind with some affinity to the target may be washed or otherwise partitioned from the remainder of the library, which may have a given level of binding affinity to the target. Washing and/or partitioning may in general include any appropriate method and/or mechanism of separating non-binding molecules, such as, for example, agitation, aspiration, flushing, and/or any other appropriate method, mechanism, or combination thereof. Flushing and/or otherwise employing a fluid for washing may generally utilize the same or similar fluid as the fluid utilized as the binding environment. The process may be repeated to partition the strongest binding members of the library. Binding may generally refer to forming a molecular complex, chemical bond, physical attachment and/or any other general intermolecular association, interaction and/or attachment. Also in general, the separating force of the washing and/or partitioning method or mechanism may generally set at least a partial threshold of binding affinity for an nucleic acids that may remain after the washing and/or partitioning step. Amplification, such as by PCR and/or other appropriate nucleic acid amplification methods, of the binding library members may also be utilized to increase the numbers of the binding members of the library for subsequent repetitions and for isolation and/or purification of any final products of the process. Embodiments of the SELEX method may generally be utilized to achieve the generation of functional biomolecules of a given binding affinity and/or range of binding affinity. The various steps of SELEX and related protocols or modifications thereof may be performed in general, utilizing appropriate conditions, such as, for example, an appropriate buffer and/or binding environment, which may be, for example, the same or similar to an environment where generated aptamers may be utilized. For cell receptor molecules, an appropriate physiological buffer and/or environment may generally be utilized for SELEX protocols. Collection of product aptamers may be achieved by, for example, elution of the nucleic acids utilizing an unfavorable environment or buffer for binding to the target, such as, for example, high osmolarity solution, which may in general interfere with binding ability of the nucleic acids. Any other appropriate collection method may also be utilized. Details of a basic SELEX protocol may be found in U.S. Pat. No. 5,270,163, entitled “Methods for identifying nucleic acid ligands,” the entire contents of which are hereby incorporated by reference. Other SELEX protocols that may generally be utilized and/or modified for an appropriate usage include those found in U.S. Pat. No. 5,789,157, entitled “Systematic evolution of ligands by exponential enrichment: tissue selex,” the entire contents of which are hereby incorporated by reference.

The SELEX technique may begin with a large library of random nucleotides or aptamers. The library may then be contacted with a target and the aptamers bound to the target may be separated and amplified for the next round. The binding conditions for each round may be made more stringent than in the previous round until the only remaining aptamers in the pool are highly specific for and bind with high affinity to the target. While aptamers may be analogous to antibodies in their range of target recognition and variety of applications, they may also possess several key advantages over their protein counterparts. For example, they are generally smaller, easier and/or more economical to produce, are capable of greater specificity and affinity, are highly biocompatible and non-immunogenic, and/or can easily be modified chemically to yield improved properties, for example, any desired properties. After selection, the selected aptamers may also be produced by chemical synthesis, which may aid in eliminating batch-to-batch variation which complicates production of therapeutic proteins.

In some exemplary embodiments, SELEX may be performed to generate aptamers utilizing a whole-cell and/or tissue approach. This may be desirable as whole-cell and/or tissue targets may present appropriate target molecules in a “native” state, such as living target cells with active and/or operative target molecules. In some embodiments, non-whole-cell targets may also be utilized, which may include, but are not limited to, purified molecular samples, anchored target molecules, artificial micelles and/or liposomes presenting target molecules, and/or any other appropriate target.

In an exemplary embodiment, biomimetic nucleic acids may be selected utilizing a SELEX protocol which may include at least one selective displacement step. For example, candidate aptamers which may be bound to a target may be selectively displaced utilizing a competitive molecule. In general, a competitive displacement may utilize a molecule with at least a similar binding affinity to the target such that equilibrium of the system may generally cause some of the bound nucleic acids to detach and be replaced by the competitive molecule. In one embodiment, aptamers may be selected for binding activity to a receptor molecule utilizing a native ligand for the receptor to selectively displace aptamers bound to the receptor molecule, such as, for example, in the active site of the receptor molecule. This may be useful, for example, to aid in selecting aptamers for agonist or antagonist activity.

In general, a library of nucleic acids may be applied to a target sample which may include receptor molecules and/or molecules which may bind to at least a particular ligand. The members of the library of nucleic acids that do not bind may be washed or partitioned from the binding members. In general, the remaining members of the library may then be bound to the target molecule. Biomimetic, agonist and/or antagonist nucleic acids may generally be members of the library that may be disruptive to normal native ligand binding to the target molecule. This may include, but is not limited to, binding to the native ligand binding site of the target, binding to the native ligand in a manner that disrupts binding to the target, binding to either native ligand or target in a manner that disrupts either's binding affinity for each other, and/or any other appropriate disruptive action and/or a combination thereof.

In an exemplary embodiment, an excess amount of the native ligand for a target molecule may then be utilized to compete off members of the library bound to the native ligand binding site or epitope of the target. In general, an excess may include an amount at or above the stoichiometric amount of available binding sites of the target(s). An excess may also be utilized as equilibrium may generally cause more of the nucleic acids to be displaced and/or dissociated. This may be desirable as it may generally dissociate members of the library bound to that particular site or epitope while generally not dissociating others. This may also be useful as members of the library dissociated by the excess native ligand may generally have a similar binding affinity to the target molecule as the native ligand. In general, binding affinity of a native ligand may be involved in its function with respect to a receptor and it may be generally useful to identify candidate biomimetic nucleic acids that may have at least similar binding affinity as this may be an indication of similar functional activity, which may be determined utilizing comparison assays. It may be the case that nucleic acids generated may have a binding affinity so high relative to the native ligand that they may not be substantially displaceable. These may be utilized as, for example, permanent and/or suicide binders, such as “always-on” agonists or suicide antagonists or inhibitors. Displacement of such molecules for collection and/or analysis may require forceful methods, such as, for example, thermal denaturation, chemical and/or osmolaric denaturation and/or any other appropriate method.

In some embodiments, a library of nucleic acids may be contacted with another background material or materials prior to selection against a target. For example, in whole cell SELEX for a receptor, a library may be contacted with cells which may not express and/or may underexpress the receptor. The binding members of the library may then be partitioned and the non-binding members may be utilized against the target receptor for SELEX. This may be useful to, for example, reduce false-positives and aid in ensuring that only binders to the desired receptor are acquired during SELEX. In general, any background material(s) may be utilized to, for example, initially screen out undesired members of a library that may bind the background material(s).

In another embodiment, members of a library may be selected for selective binding affinity to particular features of a target molecule. For example, a heterologous target molecule, such as a heterologous receptor, may be utilized as a target. The library may first be contacted with a similar target molecule, such as, for example, a wild-type and/or non-heterologous version of the molecule. Members of the library that bind may then be partitioned and the non-binders may be utilized for selection against the heterologous target molecule. This may generally be utilized with a target where there are similar molecules that may be utilized as background materials for pre-screening the library. This may be useful where the target and similar molecules may share similar molecular features and the feature of interest on the target may be unique.

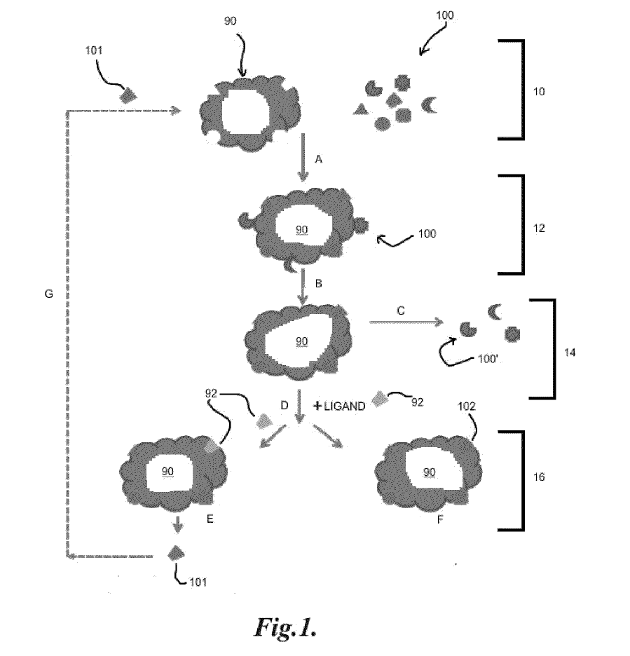

FIG. 1 illustrates an exemplary embodiment of generating biomimetic nucleic acids. A target, such as, for example, a whole cell, 90 may be contacted A with a library of nucleic acids 100 at step 10. At step 12, members of the library may be bound to the target 90. Substantially non-binding and non-binding members 100′ of the library may be washed and/or otherwise separated B and partitioned C at step 14. A native ligand and/or other molecule 92 that may naturally bind to a target of interest on the target 90 may then be added D, for example, in excess, at step 16. In case F, members of the library 102 may be bound to the target 90 so strongly that the native molecule 92 may not displace them. These members may, for example, be excluded from further rounds of selection. In case E, the native molecule 92 may displace members of the library 101, which may be collected G and utilized in subsequent rounds of selection. Resulting nucleic acids after a given number of rounds of selection may be screened for functional activity, such as, for example, agonist and/or antagonist activity against a target 90. This may, for example, be screening in comparison to the native molecule 92.

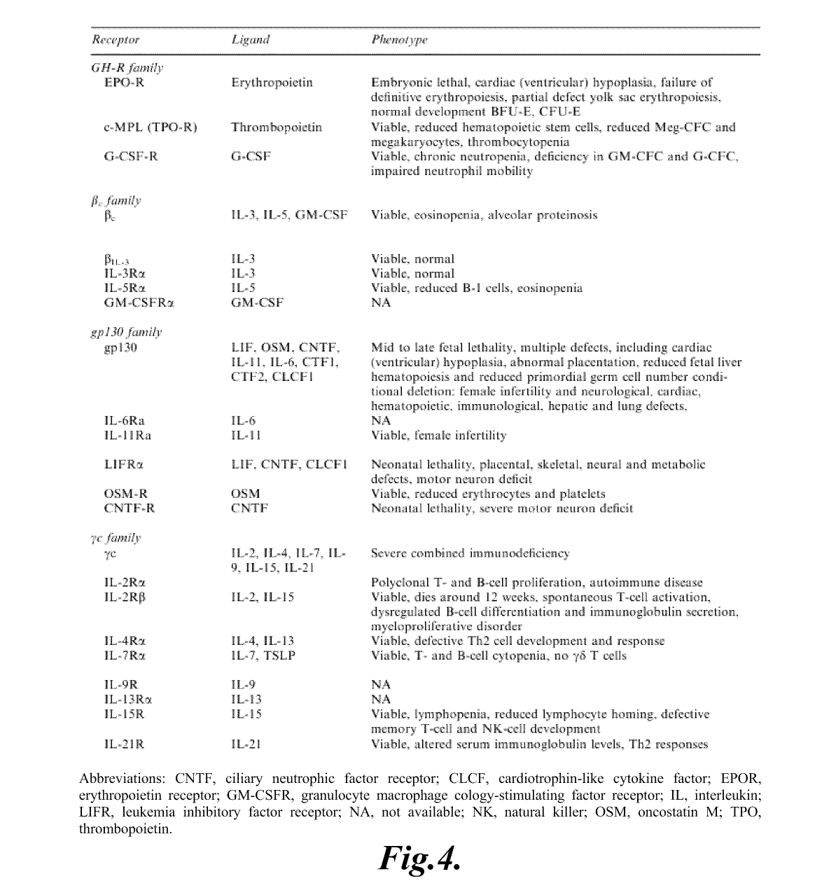

In one embodiment, biomimetic nucleic acids may be utilized to substantially mimic the activity of Stem Cell Factor (SCF), otherwise known as KIT ligand, c-KIT ligand or Steel factor, which is a cytokine which binds CD117 (c-Kit). SCF may be a growth factor important for the survival, proliferation, and differentiation of hematopoietic stem cells and other hematopoietic progenitor cells. SCF, along with a basic fibroblast growth factor (bFGF) and lymphocyte inhibitory factor (LIF), has been show to prevent spontaneous differentiation of primitive embryonic stem cells in cell culture. Biomimetic nucleic acids that mimic the activity of SCF and/or other signaling molecules may be utilized to maintain stem cells in culture without the prohibitively large cost of protein signaling molecules. This may be useful, for example, to maintain stem cells in culture in a substantially undifferentiated state utilizing biomimetic nucleic acids in the place of at least one signaling molecule. The undifferentiated stem cells may then be propagated for utilization. In one embodiment, stem cells from a patient may be preserved in a substantially undifferentiated state for potential use by the patient at a later time. Biomimetic nucleic acids may also be utilized, for example, to promote the differentiation and/or propagation of stem cells into desired lineages and/or cell types. Examples of receptor-ligand pairs which may be utilized are shown in the table of FIG. 4. It may be understood that the list is not exhaustive and any appropriate receptor-ligand and/or other molecule-binding pair may be utilized in the embodiments of the invention.

In some exemplary embodiments, biomimetic nucleic acids may be generated against multiple target molecules for a cell, tissue and/or other appropriate target material. For example, multiple signaling pathways may be affected by a mixture or “cocktail” of biomimetic nucleic acids. In general, many cellular processes and/or states may be regulated and/or affected by multiple signaling mechanisms. For example, a cocktail may be provided for maintaining stem cells in an undifferentiated state, such as with biomimetic nucleic acids mimicking the activities of SCF, thrombopoietin, and/or Flt3 ligand. In general, any combination of signaling molecules may be utilized as the basis of generating a cocktail of biomimetic nucleic acids. In some embodiments, multimeric or chimeric aptamers may be generated which may include multiple binding sites for at least one target. For example, a chimeric aptamer may be generated from two or more aptamers joined by a linking sequence which may include, for example, an oligonucleotide sequence or other polymeric linkage. In some embodiments, multimeric aptamers may be generated utilizing, for example, rolling circle amplification, such as from a circular DNA template, and/or any other appropriate method. A chimeric aptamer may, for example, be utilized to bind multiple targets in the target, such as, for example, multiple receptor molecules. In some embodiments, biomimetic nucleic acids may also be generated which may mimic multiple native ligands. This may be useful as a single aptamer may be utilized to associate with multiple target molecules.

The following examples were carried out as exemplary illustrations of the present invention and are not to be construed to be limiting in any manner.

### EXAMPLES

### 1. Generating Kit Agonist Nucleic Acids Via SELEX

To select an SCF-mimetic agonist aptamer using SELEX, as illustrated in FIG. 1a, an aptamer library initially was screened against a cell line, EML clone 1, expressing receptors for SCF (kit). During the initial SELEX process, aptamers binding to a variety of cell surface structures were selected before non-specifically bound and low affinity aptamers were substantially eliminated from the pool. After the first two rounds of selection, the cells with bound aptamers were incubated with an excess of SCF to predominantly displace kit-bound aptamers into the medium where they can be collected. This displacement step helped to ensure that at least some of the aptamers would have binding affinities substantially similar to native SCF, which was useful in selecting aptamers with the desired kit agonist activity. Aptamers having very high affinities acted as antagonists, trigger receptor desensitization or internalization due to persistent occupation of the receptors. The SCF-displaced aptamers were then cloned and sequenced (for example, approximately 50-100 sequences adequately sample the selected aptamer sequence space following 10-15 rounds of selection) and unique sequences were characterized in saturation binding experiments to determine the dissociation constants of each selected aptamer. Aptamers with affinities substantially similar to those published for SCF ranging from 40-100 pM were used in screening for agonist activity.

### 2. Example of Aptamer Library

A combinatorial DNA library containing a core randomized sequence of 40 nucleotides flanked by two 20 nucleotide conserved primer binding sites, 5′-agatagtaagtgcaatct-3′ and 5′-ataccagcttattcaatt-3′, was used as the starting library. The primers (up to 3 mismatches) were also be evaluated against the target cell genome using BLAST to insure that amplification of an endogenous gene will not occur (although this is extremely unlikely). Such a library was expected to contain approximately 10¹⁵ unique sequences. The primer binding sites were used to amplify the core sequences during the SELEX process. The 3′-primer was labeled with a purification handle, such as biotin, such that, following the PCR, the dsDNA could be purified, such as by binding to streptavidin-coated beads, to separate the antisense strand from the sense strand prior to the next round of SELEX. Other purification handles, such as aptamers, magnetic particles/nanoparticles, and/or any other appropriate affinity handle could be utilized.

### 3. Example Protocol for SELEX

The single stranded DNA pool dissolved in binding buffer was denatured by heating for 5 min at 95° C., cooled on ice for 10 min and incubated with approximately 10⁶ EML cells for 30 min at 37° C. The cells were then washed in wash buffer, centrifuged and the bound DNAs were eluted by heating at 95° C. in elution buffer. Subsequently, the eluted DNA were recovered, desalted, and amplified by PCR with a biotin-labeled 3′-primer. This allowed the separation of the selected sense ssDNA from the biotinylated antisense ssDNA by streptavidin-coated Sepharose beads for use in the next round. The process was repeated for 15 rounds. In order to increase stringency throughout the SELEX process, the washes were gradually increased in volume (1-6 ml) and duration (1-5 min) In addition, after the 3rd, 9th and 15th round, the procedure was modified in order to introduce a strong selection for kit binding, SCF-displaceable aptamers. The EML cells were rapidly washed to remove non-specifically bound aptamers, and then resuspended in binding buffer with 16.7 nM SCF. The excess SCF displaced at least some of the kit-bound aptamers so they were collected in the supernatant after centrifugation.

The aptamers after round 15 were cloned and sequenced and unique sequences were identified. The binding affinities of each of the most representative SCF-displaceable aptamers were characterized by a saturation binding assay to determine dissociation constants for each of the selected aptamers. The dissociation constants were used as guidelines for choosing the dosage ranges for measuring the cellular response to the SCF-mimetic aptamers. For the binding assay, increasing concentrations of each aptamer were incubated with a constant number of cells in binding buffer in a 96 well filter plate. After 30 minutes, the cells with bound aptamers were collected on the filter by applying a vacuum to the plate. The cells were then be rapidly washed with wash buffer as in round 15 and the bound aptamers eluted by filling each well with elution buffer and heating at 95° C. for 5 min. The eluted aptamers were quantified by qPCR. Non-specific binding was measured for each aptamer concentration in a parallel reaction containing a 1000× molar excess of the unselected ssDNA library. This represented approximately 310 ng (16.7 pmol) of SCF based on binding using ˜10⁶ cells and an assumption of ˜10,000 receptors per cell. The specific binding was defined as the difference between the total bound aptamers and the aptamer binding in the presence of the unselected library. The dissociation constants were determined by fitting the data to the equation: B=Bmax(L)/Kd+L [20], where B was the quantity of aptamer specifically bound, Bmax was the specific binding of the aptamer at saturation, L was the concentration of aptamer and Kd was the dissociation constant.

During the SCF displacement portion of the SELEX process it was possible that some non-kit-binding aptamers dissociated and appeared in the supernatant with the SCF-displaced aptamers. While it was unavoidable that a small number of non-kit-binding aptamers was selected, these were eliminated in the agonist activity screen.

As indicated above, ˜100 cloned aptamer sequences were obtained at the end of the SELEX procedure that were expected to cluster into a just a few distinct sequence motifs. To discover potential sequence motifs, all the aptamer sequences generated by the cell SELEX procedure were aligned by ClustalW as implemented in the program BioEdit. Based on the number of sequence motifs recovered two possibilities were considered:

1) After 15 rounds of SELEX, the affinity of kit-binding aptamers was so high that no aptamers were displaced by excess SCF. If this was the case, SCF-displaced aptamers from earlier rounds of SELEX would be utilized.

2) The aptamer-receptor complex underwent internalization during the incubation such that the aptamers were not recovered easily. To test for this possibility, the cells were extensively washed after the incubation to remove all cell surface-bound aptamers, then the cells are lysed and the lysate tested for the presence of aptamers by qPCR. Aptamers in the lysate indicated internalization and the incubation temperature was lowered to aid blocking internalization as in some published cell SELEX protocols.

### 4. Example Protocol for Agonist Activity Determination

EML cells were seeded into 96 well culture plates at 3×10⁴ cells/well in standard media without SCF and placed in a humidified CO₂ incubator at 37° C. Aptamers from a SELEX protocol, as above, were added to the wells at concentrations equal to the measured Kd values, the concentration of aptamer at which the receptors are 50% occupied. A set of control wells with and without 20 ng/ml SCF was included (20 ng/ml is the concentration of SCF recommended by ATCC for culture of EML cells). The EML cells were cultured until the SCF(+) control reached approximately 80% confluence. At that point, all the wells were assayed for cell number by MTT (Vybrant MTT Cell Proliferation Assay, Invitrogen). Aptamers which stimulate cell proliferation above the SCF(−) control were considered SCF-mimetic agonists.

While the SELEX procedure allows the selection of aptamers with binding affinities similar to SCF, it has no bearing on the efficacy of each molecule. Efficacy is defined here as the ability of a bound ligand to cause the receptor to undergo a conformational change, activating a signaling pathway resulting in a physiological response. It is well established that ligands having similar binding affinities for a receptor can produce different degrees of response.

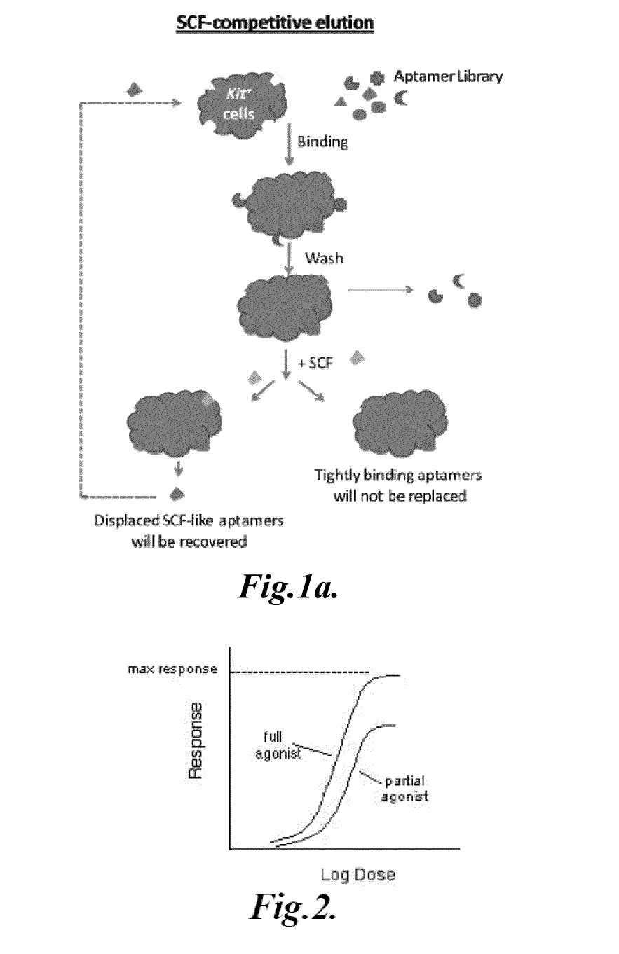

In the initial screening of selected aptamers only a single concentration of aptamer was used to test agonist activity. To determine which, if any, aptamers had agonist activity, a log dose-response curve was produced for each aptamer showing SCF-mimetic activity in the initial screen. The protocol used was the same as in the initial agonist screening except that increasing doses of agonist aptamers and SCF were incubated in each well. SCF was presumed to be a full agonist. FIG. 2 shows a dose response curve. Each dose-response experiment was repeated 5 times. The final product was aptamers which were at the very least partial SCF-agonists and at best full SCF-agonists.

From all dose-response curves, the mean of the maximum responses (i.e. the cell numbers as measured by the MTT assay) for SCF and agonist aptamers was calculated. The maximum response was defined as the same response occurring at two successive doses. The mean maximum response of each aptamer was compared to the control SCF response by one way ANOVA and Dunetts test, (p<0.05). Aptamers with maximum responses not statistically different (i.e. not rejecting the null hypothesis) from SCF were considered full agonists. If none of the aptamers with SCF-mimetic activity were full agonists, derivatives of the most highly conserved motifs were tested to see if small differences in nucleotide composition improved biological activity.

One may exhaustively investigate such sequence motifs “leads” for full-agonist activity by ordering customized cocktails of oligonucleotides synthesized on microarrays (LC Sciences/Atactic, Houston, Tex., for example). For example, if a particular sequence motif within the randomized oligonucleotide region emerges as a promising kit binder, using microarray technology, approximately 10⁵ to 10⁶ derivatives of these oligos may be readily created on a microarray and amplified for further study. While a completely randomized library may be readily purchased in a single tube, the microarray approach for synthesis of a library of reduced complexity for investigating leads may be useful for this purpose.

### 5. Example of Fold Differences in Cell Proliferation

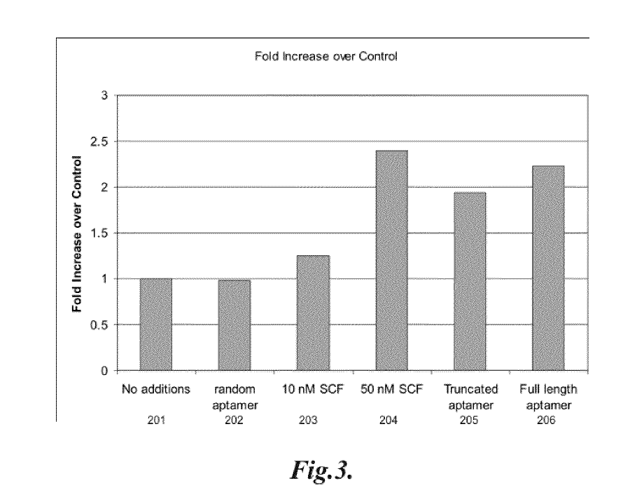

EML cells were cultured for 5 days and then measured for cell proliferation based on total DNA. FIG. 3 shows the fold-increase over control of cell proliferation of EML cells incubated with no additions (201), addition of a randomized aptamer (202), 10 nM SCF (203), 50 nM SCF (204), a first biomimetic aptamer (205), which was a DNA aptamer having the sequence of 5′-ataccagcttattcaattGGCAAGGGGTAGACACGCGGCGCGGGACCGGGAGCCGACAa gatagtaagtgcaatct-3′, and a second biomimetic aptamer (206), which was a DNA aptamer having the sequence of 5′-ataccagcttattcaattGGCCAGGCACTAACTAGTTGGCCGCATTAAAGACCTAATGa gatagtaagtgcaatctATACGAGCGTGATTATCAATCCTCGTACACCGGGTACTGGAA TTGAATAAGCTGGTAT-3′. As shown, no addition 201 and the addition of random aptamer 202 produced substantially no increase in proliferation over the control. A 10 nM SCF (203) produced a 1.25 fold increase while a 50 nM SCF (204) produced a 2.40 fold increase over the control. The first aptamer 205 produced a 1.94 fold increase and the second aptamer 206 produced a 2.23 fold increase. This illustrates the biomimetic properties of the aptamers produced with embodiments described above.

It will be appreciated by those of ordinary skill in the art that the present invention can be embodied in other specific forms without departing from the spirit or essential character hereof. The present description is therefore considered in all respects to be illustrative and not restrictive. The scope of the present invention is indicated by the appended claims, and all changes that come within the meaning and range of equivalents thereof are intended to be embraced therein.

1. A method for generating biomimetic nucleic acids comprising:
a) contacting a library of nucleic acids with a target molecule;
b) partitioning non-binding members to said target molecule of said library from binding members to said target biomolecule of said library;
c) selectively displacing the binding member to said target biomolecule utilizing at least a molecule that natively binds to said target molecule;
d) collecting displaced binding members of the library;
e) applying said displaced binding members of the library from step d to said target and repeating steps c and d for a selected number of rounds of selection; and
f) screening members of the library remaining from said number of rounds of selection for functional activity against said target molecule in comparison to said molecule that natively binds to said target.

2. The method of claim 1, wherein said functional activity comprises agonist or antagonist activity.

3. The method of claim 1, wherein said target molecule is a cell signaling receptor.

4. The method of claim 3, wherein said natively binding molecule is a ligand for said cell signaling receptor.

5. The method of claim 1, further comprising amplifying the displaced binding members between steps d and e.

6. The method of claim 1, further comprising contacting said library with a background material and collecting the non-binding members of the library for contacting with said target.

7. A functional ligand comprising:
a nucleic acid which binds with specificity to a target molecule, said nucleic acid is generated by a selective propagation method and is selectively displaceable from said target molecule by another molecule; wherein said functional ligand has a functional activity in relation to said target molecule.

8. The functional ligand of claim 7, wherein said target molecule comprises a cell signaling receptor.

9. The functional ligand of claim 8, wherein said another molecule comprises a native ligand to said receptor.

10. The functional ligand of claim 8, wherein said functional activity comprises agonist or antagonist activity.

11. The functional ligand of claim 7, further comprising an affinity handle.

12. The functional ligand of claim 8, wherein said receptor comprises a stem cell factor receptor.

13. The functional ligand of claim 12, wherein said target molecule comprises stem cell factor.

14. The functional ligand of claim 9, wherein said nucleic acid substantially mimics the functional activity of said native ligand.

15. A method for culturing cells comprising:
a) contacting a library of nucleic acids with target cells;
b) partitioning non-binding members of said library from binding members of said library;
c) selectively displacing binding members of the library utilizing at least a molecule that natively binds to a cell-signaling receptor of said target cells;
d) collecting displaced members of the library;
e) applying displaced members of the library to said target cells and repeating steps c and d for a selected number of rounds of selection;
f) screening members of the library remaining from the said number of rounds of selection for functional activity against said target cells in comparison to said natively binding molecule; and
g) culturing target cells utilizing at least one screened member of said library to apply said functional activity to said target cells.

16. The method of claim 15, wherein said target cells comprise stem cells.

17. The method of claim 16, wherein said cell-signaling receptor comprises stem cell factor receptor.

18. The method of claim 17, wherein said natively binding molecule comprises stem cell factor.

19. The method of claim 18, wherein screening for functional activity comprises comparing the functional activity of the displaced members against the activity of stem cell factor.

20. The method of claim 15, further comprising amplifying displaced members of said library between steps d and e.

21. The method of claim 16, wherein said functional activity comprises maintaining a substantially undifferentiated state of said stem cells.

22. The method of claim 15, further comprising contacting said library with a background material and collecting the non-binding members of the library for contacting with said target cells.


SEQUENCE LISTING (6 sequences)

SEQ ID NO: 1 - DNA, 76 bp, Mus musculus
ataccagcttattcaattggcaaggggtagacacgcggcgcgggaccgggagccgacaagatagtaagtgcaatct

SEQ ID NO: 2 - DNA, 76 bp, Mus musculus
agatagtaagtgcaatctgttaagtttgactataacaacccggacctgttattcggggaattgaataagctggtat

SEQ ID NO: 3 - DNA, 134 bp, Mus musculus
ataccagcttattcaattggcaaggggtagacacgcggcgcgggaccgggagccgacaagatagtaagtgcaatctgttaagtttgactataacaacccggacctgttattcggggaattgaataagctggtat

SEQ ID NO: 4 - DNA, 76 bp, Mus musculus
ataccagcttattcaattggccaggcactaactagttggccgcattaaagacctaatgagatagtaagtgcaatct

SEQ ID NO: 5 - DNA, 76 bp, Mus musculus
agatagtaagtgcaatctatacgagcgtgattatcaatcctcgtacaccgggtactggaattgaataagctggtat

SEQ ID NO: 6 - DNA, 134 bp, Mus musculus
ataccagcttattcaattggccaggcactaactagttggccgcattaaagacctaatgagatagtaagtgcaatctatacgagcgtgattatcaatcctcgtacaccgggtactggaattgaataagctggtat